(19) United States
(12) Patent Application Publication
(10) Pub. No.: US 2020/0190594 A1
(43) Pub. Date: Jun. 18, 2020

(54) INVESTIGATING TUMORAL AND TEMPORAL HETEROGENEITY THROUGH COMPREHENSIVE -OMICS PROFILING IN PATIENTS WITH METASTATIC TRIPLE NEGATIVE BREAST CANCER

(71) Applicant: NANTOMICS, LLC, Culver City, CA (US)

(72) Inventors: Christopher SZETO, Culver City, CA (US); Rahul PARULKAR, Culver City, CA (US); Andrew SEDGEWICK, Culver City, CA (US)

(21) Appl. No.: 16/617,844

(22) PCT Filed: May 31, 2018
(86) PCT No.: PCT/US2018/035410

§ 371 Date: Nov. 27, 2019

Related U.S. Application Data
(60) Provisional application No. 62/513,942, filed on Jun. 1, 2017.

(51) Int. Cl. C12Q 1/6886; G01N 33/574; G16B 5/00; G16B 20/20

(52) U.S. Cl. CPC C12Q 1/6886; G01N 33/57415; G16B 5/00; C12Q 2600/106; C12Q 2600/118; C12Q 2600/156; G16B 20/20

(57) ABSTRACT

Molecular profiles of metastatic tumors can be more accurately determined using combination of DNA mutational profiles and RNA expression profiles of selected genes that show substantial changes upon anti-tumor treatment. Such combinatorial information can be used to determine differential pathway activity that may be related to the sensitivity or resistance to the anti-tumor treatment.

This application claims priority to our co-pending US provisional application with the Ser. No. 62/513,942, filed Jun. 1, 2017.

### FIELD OF THE INVENTION

The field of the invention is comprehensive characterization of molecular profiles of a metastatic tumor using omics analysis of cancer patients.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

One of the most significant challenges in treating cancer is individual and/or tumor-specific variance in responding to a tumor treatment. While many determinants may affect such individual or tumor variances, many studies point to intrinsic sensitivity/resistance of the tumor as a major factor, which may be determined by identifying and analyzing molecular profile of the tumor. With the advent of whole genome sequencing and next generation sequencing platforms, massive quantities of data are now available for molecular profile analysis. While the wealth of data is certainly desirable from a theoretical perspective, it is not often clear what types and/or which data from the massive quantities of data are significantly relevant to determine the sensitivity/resistance to the tumor treatment, and how such data should be analyzed together to determine accurate molecular profile of the tumor with respect to the tumor treatment.

To circumvent such difficulties, efforts have been made to use selected omics data to determine a molecular profile of a tumor. For example, US Patent Publication No. 2010/0035257 to Ellisen discloses that a tumor that is responsive to platinum-based chemotherapy can be identified by determining the quantity and/or activity of P63 isoform and/or p73 isoform. In another example, US Patent Publication No. 2014/0162887 to Martin discloses determination of prognosis of cancer using expression level of a plurality of marker gene, including CKS2, CDKN3, FOXM1, RRM2, etc. In still another example, US Patent Publication No. 2016/0122827 to Szallasi discloses that effective therapy for a tumor can be selected by determining expression level of BML and FANCI genes and/or copy number in the chromosome location 15q26. However, all of those are limited to analyzing piecemeal information that evaluates how individual marker genes are expressed and/or an existence of a specific mutation in the chromosome. As such, all those fail to use omics data to determine systemic changes in the tumor, which is a key attribute of intrinsic sensitivity and/or resistance to a tumor treatment.

Thus, even if several common mutations and/or expression levels of marker genes in the tumor are known to be relevant to sensitivity and/or resistance to chemotherapeutic drugs, most omics analyses to determine the sensitivity and/or resistance to chemotherapeutic drugs and/or prognosis of the tumor suffer from disadvantages, including heavy reliance on a few markers that may not have substantial physiological impact on drug sensitivity/resistance of the tumor cell, and inability to take account of many factors that are not mutated or overexpressed, yet play a key role in regulating drug sensitivity/resistance in a signaling pathway, in which some marker genes play as elements. Therefore, there remains a need for improved methods and systems to use omics data for comprehensive characterization of molecular profiles of a metastatic tumor.

### SUMMARY OF THE INVENTION

The inventive subject matter is directed to various methods for using various types of omics data for comprehensive characterization of molecular profiles of a metastatic tumor by reconstructing or modifying signaling pathways of the tumor cells in silico by incorporating various, yet selected, omics data that are relevant to drug sensitivity/resistance. Thus, one aspect of the inventive subject matter includes method of predicting prognosis of a tumor in a patient upon an anti-tumor treatment. This method comprises a step of obtaining a tumor sample from the patient and an omics data set from the tumor sample and determining a genomic mutation profile from the omics data set and an RNA expression profile of a plurality of genes. Preferably, the genomic mutation profile comprises a mutation burden of a plurality of somatic mutations. The method continues with a step of obtaining a pathway model comprising a plurality of pathway elements. Then, a modified pathway activity can be inferred by integrating the genomic mutation profile and the RNA expression profile into the pathway model, at least one of which is related to at least one of the pathway elements. The modified pathway activity is an indicative of a response to the anti-tumor treatment that may include, but not limited to, sensitivity, unresponsiveness, and acquired resistance to the treatment.

Most typically, genomic mutation profile is obtained from a whole genome sequencing, an exome sequencing, or RNAseq, and the plurality of somatic mutations identified in the genomic mutation profile comprises a point mutation, an amplification, a deletion, and an insertion. In addition, the genomic mutation profile further comprises a copy number of a genome segment having the somatic mutation. Further, the mutational burden by the plurality of somatic mutations is determined by number of the plurality of somatic mutations occurring in a predetermined time period.

In preferred aspects, at least two of the plurality of genes for RNA expression profile analysis includes RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6. Preferably, at least two of the plurality of genes are involved in at least one of a signaling pathway regulating pluripotency of stem cells, a cancer signaling pathway, and a pyrimidine metabolism. Of course, it should be appreciated that the above genes may be wild type or mutated versions, including missense or nonsense mutations, insertions, deletions, fusions, and/or translocations.

With respect to the modified pathway activity, it is contemplated that the modified pathway activity is at least one of modified protein expression level and modified post-translational modification of at least one pathway element. In some embodiments, the modified pathway activity comprises a pathway element in MYC/MAX pathway, p53 pathway or ETS1 pathway. Preferably, the modified pathway activity is inferred using PARADIGM pathway analysis.

Additionally, the method may further comprise a step of measuring a protein activity from the tumor sample and comparing the protein activity with the modified pathway activity. In such method, the protein activity comprises at least one of quantity of the protein and a post-translational modification of the protein. Where the measured protein activity is different from the modified pathway activity or provides additional information to the modified pathway activity, the protein activity can be integrated to pathway model to refine the modified pathway activity. Finally, such modified pathway activity can be used to generate or update the patient's record.

In yet another aspect of the inventive subject matter, the inventors contemplate a method of predicting treatment efficacy of an anti-tumor treatment on a metastatic tumor in a patient. In this method, a plurality of tumor sample from at least two different anatomical locations in the patient and respective omics data sets from each of the tumor sample are obtained. Then, respective genomic mutation profiles from the omics data sets and RNA expression profiles of a plurality of genes are determined from the omics data sets and/or from each of the tumor sample. Preferably, the genomic mutation profile comprises a mutation burden of a plurality of somatic mutations. The method continues with a step of obtaining a pathway model comprising a plurality of pathway elements. Then, a modified pathway activity can be inferred by integrating the genomic mutation profile and the RNA expression profile into the pathway model, at least one of which is related to at least one of the pathway elements. The modified pathway activity is an indicative of a response to the anti-tumor treatment that may include, but not limited to, sensitivity, unresponsiveness, and acquired resistance to the treatment. Then, the method continues with generating or updating the patient's record using the modified pathway activities. The patient's record, preferably, includes at least one of a likelihood of success of the anti-tumor treatment to treat the metastatic tumor and an updated treatment recommendation to the patient.

Preferably, the tumor is a metastatic triple-negative breast cancer, and the anti-tumor treatment is any platinum-based therapy, for example, cisplatin or cisplatin-derived treatment. Thus, it is contemplated that the plurality of tumor samples from at least two different anatomical locations are originated and/or derived from the same triple-negative breast cancer, potentially via metastasis. In some embodiments, the plurality of tumor samples are obtained before and after the anti-tumor treatment and/or are obtained during the anti-tumor treatment and/or after the anti-tumor treatment is completed. The obtained omics data from the plurality of tumor samples is compared with the omics data of matched normal tissue (e.g., healthy tissue of the patient, etc.) to identify tumor-specific alteration of omics data.

Most typically, genomic mutation profile is obtained from a whole genome sequencing, an exome sequencing, or RNAseq, and the plurality of somatic mutations identified in the genomic mutation profile comprises a point mutation, an amplification, a deletion, and an insertion. In addition, the genomic mutation profile further comprises a copy number of a genome segment having the somatic mutation. Further, the mutational burden by the plurality of somatic mutations is determined by number of the plurality of somatic mutations occurring in a predetermined time period.

In preferred aspects, at least two of the plurality of genes for RNA expression profile analysis includes RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6. Preferably, at least two of the plurality of genes are involved in at least one of a signaling pathway regulating pluripotency of stem cells, a cancer signaling pathway, and a pyrimidine metabolism. Of course, it should be appreciated that the above genes may be wild type or mutated versions, including missense or nonsense mutations, insertions, deletions, fusions, and/or translocations.

With respect to the modified pathway activity, it is contemplated that the modified pathway activity is at least one of modified protein expression level and modified post-translational modification of at least one pathway element. In some embodiments, the modified pathway activity comprises a pathway element in MYC/MAX pathway, p53 pathway or ETS1 pathway. Preferably, the modified pathway activity is inferred using PARADIGM pathway analysis.

Additionally, the method may further comprise a step of measuring a protein activity from the tumor sample and comparing the protein activity with the modified pathway activity. In such method, the protein activity comprises at least one of quantity of the protein and a post-translational modification of the protein. Where the measured protein activity is different from the modified pathway activity or provides additional information to the modified pathway activity, the protein activity can be integrated to pathway model to refine the modified pathway activity.

Still another aspect of the inventive subject matter includes a method of predicting a response to cisplatin treatment in a patient having a triple negative breast cancer. This method comprises a step of obtaining a tumor sample from the patient and an omics data set from the tumor sample and determining an RNA expression profile of a plurality of genes. Most preferably, the at least one of the plurality of genes are related to at least one of a signaling pathway regulating pluripotency of stem cells, a cancer signaling pathway, and a pyrimidine metabolism. The method continues with a step of obtaining a pathway model comprising a plurality of pathway elements. Then, a modified pathway activity can be inferred by integrating the RNA expression profile into the pathway model, at least one of which is related to at least one of the pathway elements. The modified pathway activity is an indicative of a response to the anti-tumor treatment that may include, but not limited to, sensitivity, unresponsiveness, and acquired resistance to the treatment.

In preferred aspects, at least two of the plurality of genes for RNA expression profile analysis includes RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6.

Optionally, in addition to the RNA expression profile, a genomic mutation profile can be determined from the omics data set, where the genomic mutation profile comprises a mutation burden of a plurality of somatic mutations, and a modified pathway activity can be inferred by integrating the genomic mutation profile and the RNA expression profile into the pathway model. Further, a protein activity can be measured from the tumor sample, where the protein activity comprises at least one of quantity of the protein and a post-translational modification of the protein, and the protein activity can be compared with the modified pathway activity. Such measured protein activity can be integrated to refine the modified pathway activity.

Various objects, features, aspects and advantages of the inventive subject matter will become more apparent from the following detailed description of preferred embodiments and accompanied drawings.

### BRIEF DESCRIPTION OF THE DRAWING

FIG. 1 depicts a timelines of 20 triple negative breast cancer (TNBC) patients from which biopsied tumor tissues were obtained for omics analysis.

FIG. 2A shows a bar graph depicting the high-confident genomic copy-number aberration (CNA) in individual biopsies of 17 patients.

FIG. 2B shows a bar graph depicting exomic single nucleotide variants (SNVs) and insertion and deletion of bases (INDELs) identified in individual biopsies of 17 patients.

FIG. 2C shows Venn diagrams showing counts of existing and acquired SNVs in the pre-cisplatin and post-cisplatin tumor tissues in 4 patients.

FIG. 3A shows a heat map of 57 genes that are significantly differentially expressed between cisplatin sensitive patients and resistant patients.

FIG. 3B shows a table providing that 57 genes shown in the heat map of FIG. 3A are enriched for cancer signaling and pyrimidine metabolism pathways.

FIG. 4A describes a schematic diagram of generating inferred pathway activities using PARADIGM pathway analysis.

FIG. 4B shows comparisons of observed protein amount from quantitative mass spectrometry and inferred protein activity from PARADIGM for ten proteins that discriminate cisplatin-naïve samples from cisplatin-treated samples.

FIG. 5A depicts changes in cell pathway activities between pre- and post-cisplatin treatment in 6 different biopsies from 4 patients.

FIG. 5B depicts relationships of multiple cell pathways in relation to ETS1 pathway, and depicts pathway elements whose activities are changed between pre- and post-cisplatin treatment.

### DETAILED DESCRIPTION

The inventors contemplate that genomic, transcriptomic, and/or proteomic changes observed in the tumor cells, individually or in combination, alter cell signaling pathways of tumor cells such that the tumor cells show distinct intrinsic physiological characters, for example, sensitivity or resistance to a drug, susceptibility to necrosis or apoptosis, de-differentiation and/or acquisition of stem-cell-like characters, or acquisition of metastatic characters. While identification of individual changes in DNA, transcript expression level, or protein activity may be associated with some physiological changes of the tumor cells and may be used as a marker to predict the status of the cell, such approach often fails to take account of individual variances in such changes or overall or net changes in the cell signaling networks that may lead to the similar or distinct physiological characteristics of the tumor cells. For example, tumor A and tumor B may have different expression levels in gene C, D and E (e.g., tumor A has increased expression in C and D, and tumor B has increased expression in E only) while tumor A and B possess substantially similar drug sensitivity to chemotherapeutic agent F. In another example, tumor G and H may show similar expression levels in marker genes I and J, yet possess distinct drug sensitivity to chemotherapeutic agent K because of another factors that were not identified as marker genes or proteins.

Viewed from a different perspective, the inventors discovered that various omics data obtained from the patient's tumor tissues can be collectively used to determine the overall or net changes in the cell signaling networks that can affect the intrinsic properties of the tumor tissues. In addition, a group of omics data can be selected as a relevant data set for specific physiological characteristic of the tumor cell (e.g., sensitivity to treatment A, etc.) such that the cell signaling network analysis using the omics data set can be efficiently and effectively performed in a timely manner.

Consequently, in one especially preferred aspect of the inventive subject matter, the inventors contemplate a method of predicting prognosis of a tumor in a patient upon an anti-tumor treatment using genomic mutation profile and RNA expression profile obtained from the omics data set from the tumor sample. The genomic mutation profile and RNA expression profile is integrated to a pathway model to infer a modified pathway activity due to the difference of genomic mutation profile and RNA expression profile from the matched normal. Such inferred modified pathway activity can be an indicative of a response to the anti-tumor treatment and further used to predict prognosis of a tumor in a patient upon an anti-tumor treatment. Additionally, proteomics data may also be used for such methods.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

### Obtaining Omics Data

Any suitable methods of obtaining a tumor sample (tumor cells or tumor tissue) from the patient (or healthy tissue from a patient or a healthy individual as a comparison) are contemplated. Most typically, a tumor sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue. For example, the tumor cells or tumor tissue may be fresh or frozen. For other example, the tumor cells or tumor tissues may be in a form of cell/tissue extracts. In some embodiments, the tumor samples may be obtained from a single or multiple different tissues or anatomical regions. For example, a metastatic breast cancer tissue can be obtained from the patient's breast as well as other organs (e.g., liver, brain, lymph node, blood, lung, etc.) for metastasized breast cancer tissues. Preferably, a healthy tissue of the patient or matched normal tissue (e.g., patient's non-cancerous breast tissue) can be obtained or a healthy tissue from a healthy individual (other than the patient) can be also obtained via a similar manner as a comparison.

In some embodiments, tumor samples can be obtained from the patient in multiple time points in order to determine any changes in the tumor samples over a relevant time period. For example, tumor samples (or suspected tumor samples) may be obtained before and after the samples are determined or diagnosed as cancerous. In another example, tumor samples (or suspected tumor samples) may be obtained before, during, and/or after (e.g., upon completion, etc.) a one time or a series of anti-tumor treatment (e.g., radiotherapy, chemotherapy, immunotherapy, etc.). In still another example, the tumor samples (or suspected tumor samples) may be obtained during the progress of the tumor upon identifying a new metastasized tissues or cells.

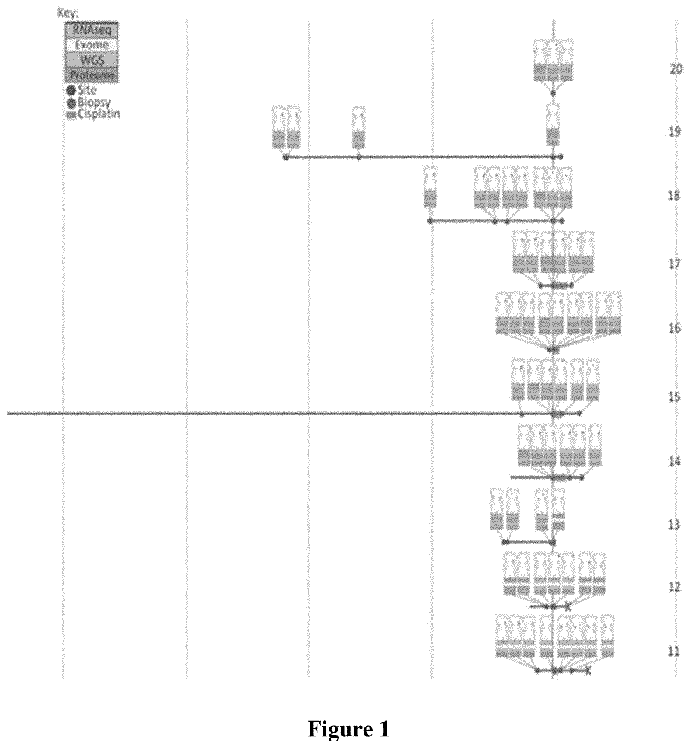

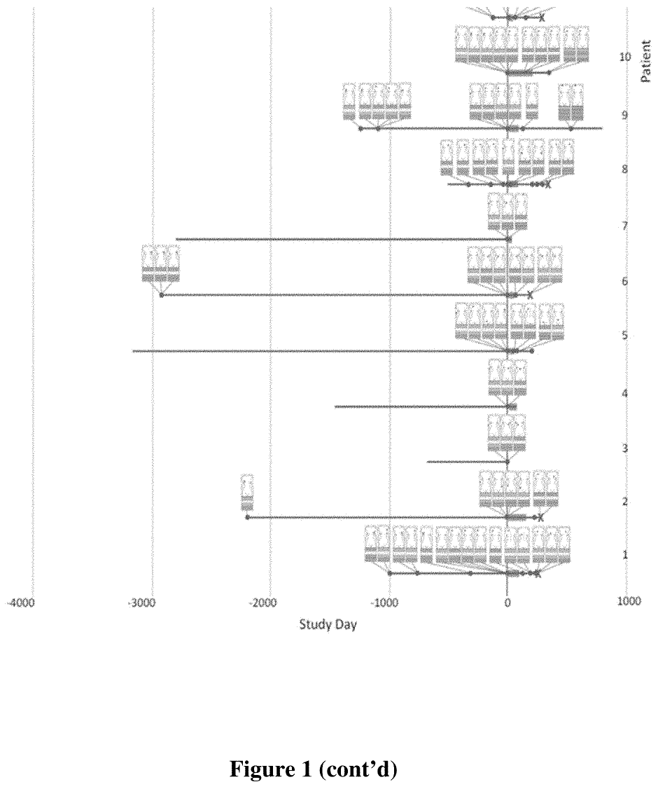

An exemplary timeline to obtain tumor sample from 20 triple negative breast cancer (TNBC) patients and/or omics data from the tumor sample is described in FIG. 1. The “Intensive Trial of OMics in Cancer”-001 (ITOMIC-001; Clinicaltrials.gov ID: NCT01957514) enrolled patients with metastatic triple negative breast cancer (TNBC) who are platinum-naive and scheduled to receive cisplatin. Multiple biopsies (up to 7 metastatic sites) were performed under carefully controlled conditions prior to and upon completion of cisplatin treatment and following any subsequent therapies. Boxes denote biopsy sites (location in the patient's body), timing, treatment with cisplatin, and types of omics data obtained. Study date 0 is the first date of inclusion on trial. Most patients shown in FIG. 1 have prior breast cancer diagnoses, thus have timelines before day 0. Many patients have multiple historical biopsies that were analyzed as part of the trial. As shown, biopsies of tumor tissues were performed in different locations in the body (e.g., left or right breast, ovary, lower abdomen, etc.) in different time points. From some of those biopsied samples, different types or combination of types of omics data were often obtained based on the type of biopsy, tissue availability, tissues status, relevant information required at a given time period, etc.

From the obtained tumor cells or tumor tissue, DNA (e.g., genomic DNA, extrachromosomal DNA, etc.), RNA (e.g., mRNA, miRNA, siRNA, shRNA, etc.), and/or proteins (e.g., membrane protein, cytosolic protein, nucleic protein, etc.) can be isolated and further analyzed to obtain omics data. Alternatively and/or additionally, a step of obtaining omics data may include receiving omics data from a database that stores omics information of one or more patients and/or healthy individuals. For example, omics data of the patient's tumor may be obtained from isolated DNA, RNA, and/or proteins from the patient's tumor tissue, and the obtained omics data may be stored in a database (e.g., cloud database, a server, etc.) with other omics data set of other patients having the same type of tumor or different types of tumor. Omics data obtained from the healthy individual or the matched normal tissue (or healthy tissue) of the patient can be also stored in the database such that the relevant data set can be retrieved from the database upon analysis. Likewise, where protein data are obtained, these data may also include protein activity, especially where the protein has enzymatic activity (e.g., polymerase, kinase, hydrolase, lyase, ligase, oxidoreductase, etc.).

As used herein, omics data includes but is not limited to information related to genomics, proteomics, and transcriptomics, as well as specific gene expression or transcript analysis, and other characteristics and biological functions of a cell. With respect to genomics data, suitable genomics data includes DNA sequence analysis information that can be obtained by whole genome sequencing and/or exome sequencing (typically at a coverage depth of at least 10×, more typically at least 20×) of both tumor and matched normal sample. Alternatively, DNA data may also be provided from an already established sequence record (e.g., SAM, BAM, FASTA, FASTQ, or VCF file) from a prior sequence determination. Therefore, data sets may include unprocessed or processed data sets, and exemplary data sets include those having BAM format, SAM format, FASTQ format, or FASTA format. However, it is especially preferred that the data sets are provided in BAM format or as BAMBAM diff objects (e.g., US2012/0059670A1 and US2012/0066001A1). Omics data can be derived from whole genome sequencing, exome sequencing, transcriptome sequencing (e.g., RNA-seq), or from gene specific analyses (e.g., PCR, qPCR, hybridization, LCR, etc.). Likewise, computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers. Such analysis advantageously reduces false positive neoepitopes and significantly reduces demands on memory and computational resources.

With respect to the analysis of tumor and matched normal tissue of a patient, numerous manners are deemed suitable for use herein so long as such methods will be able to generate a differential sequence object or other identification of location-specific difference between tumor and matched normal sequences. Exemplary methods include sequence comparison against an external reference sequence (e.g., hg18, or hg19), sequence comparison against an internal reference sequence (e.g., matched normal), and sequence processing against known common mutational patterns (e.g., SNVs). Therefore, contemplated methods and programs to detect mutations between tumor and matched normal, tumor and liquid biopsy, and matched normal and liquid biopsy include iCallSV (URL: github.com/rhshah/iCallSV), VarScan (URL: varscan.sourceforge.net), MuTect (URL: github.com/broadinstitute/mutect), Strelka (URL: github.com/Illumina/strelka), Somatic Sniper (URL: gmt.genome.wustl.edu/somatic-sniper/), and BAMBAM (US 2012/0059670).

However, in especially preferred aspects of the inventive subject matter, the sequence analysis is performed by incremental synchronous alignment of the first sequence data (tumor sample) with the second sequence data (matched normal), for example, using an algorithm as for example, described in Cancer Res 2013 Oct. 1; 73(19):6036-45, US 2012/0059670 and US 2012/0066001 to so generate the patient and tumor specific mutation data. As will be readily appreciated, the sequence analysis may also be performed in such methods comparing omics data from the tumor sample and matched normal omics data to so arrive at an analysis that can not only inform a user of mutations that are genuine to the tumor within a patient, but also of mutations that have newly arisen during treatment (e.g., via comparison of matched normal and matched normal/tumor, or via comparison of tumor). In addition, using such algorithms (and especially BAMBAM), allele frequencies and/or clonal populations for specific mutations can be readily determined, which may advantageously provide an indication of treatment success with respect to a specific tumor cell fraction or population. Thus, omics data analysis may reveal missense and nonsense mutations, changes in copy number, loss of heterozygosity, deletions, insertions, inversions, translocations, changes in microsatellites, etc.

Moreover, it should be noted that the data sets are preferably reflective of a tumor and a matched normal sample of the same patient to so obtain patient and tumor specific information. Thus, genetic germ line alterations not giving rise to the tumor (e.g., silent mutation, SNP, etc.) can be excluded. Of course, it should be recognized that the tumor sample may be from an initial tumor, from the tumor upon start of treatment, from a recurrent tumor or metastatic site, etc. In most cases, the matched normal sample of the patient may be blood, or non-diseased tissue from the same tissue type as the tumor.

In addition, omics data of cancer and/or normal cells comprises transcriptome data set that includes sequence information and expression level (including expression profiling or splice variant analysis) of RNA(s) (preferably cellular mRNAs) that is obtained from the patient, most preferably from the cancer tissue (diseased tissue) and matched healthy tissue of the patient or a healthy individual. There are numerous methods of transcriptomic analysis known in the art, and all of the known methods are deemed suitable for use herein (e.g., RNAseq, RNA hybridization arrays, qPCR, etc.). Consequently, preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis, especially including RNAseq. In other aspects, RNA quantification and sequencing is performed using RNA-seq, qPCR and/or rtPCR based methods, although various alternative methods (e.g., solid phase hybridization-based methods) are also deemed suitable. Viewed from another perspective, transcriptomic analysis may be suitable (alone or in combination with genomic analysis) to identify and quantify genes having a cancer- and patient-specific mutation.

It should be appreciated that one or more desired nucleic acids may be selected for a particular disease, disease stage, specific mutation, or even on the basis of personal mutational profiles or presence of expressed neoepitopes. Alternatively, where discovery or scanning for new mutations or changes in expression of a particular gene is desired, real time quantitative PCR may be replaced by RNAseq to so cover at least part of a patient transcriptome. Moreover, it should be appreciated that analysis can be performed static or over a time course with repeated sampling to obtain a dynamic picture without the need for biopsy of the tumor or a metastasis.

Further, omics data of cancer and/or normal cells comprises proteomics data set that includes protein expression levels (quantification of protein molecules), post-translational modification, protein-protein interaction, protein-nucleotide interaction, protein-lipid interaction, and so on. Thus, it should also be appreciated that proteomic analysis as presented herein may also include activity determination of selected proteins. Such proteomic analysis can be performed from freshly resected tissue, from frozen or otherwise preserved tissue, and even from FFPE tissue samples. Most preferably, proteomics analysis is quantitative (i.e., provides quantitative information of the expressed polypeptide) and qualitative (i.e., provides numeric or qualitative specified activity of the polypeptide). Any suitable types of analysis are contemplated. However, particularly preferred proteomics methods include antibody-based methods and mass spectroscopic methods. Moreover, it should be noted that the proteomics analysis may not only provide qualitative or quantitative information about the protein per se, but may also include protein activity data where the protein has catalytic or other functional activity. One exemplary technique for conducting proteomic assays is described in U.S. Pat. No. 7,473,532, incorporated by reference herein. Further suitable methods of identification and even quantification of protein expression include various mass spectroscopic analyses (e.g., selective reaction monitoring (SRM), multiple reaction monitoring (MRM), and consecutive reaction monitoring (CRM)).

### Omics Data Analysis

The inventors contemplate that a molecular profile or a molecular signature of the tumor tissue can be determined using omics data, preferably two or more types of omics data. While any types or subtypes of omics data may be used to determine the molecular profile or a molecular signature of the tumor tissue, it is contemplated that the type of omics data preferred may differ based on the type of tumor, based on the desired information (e.g., information on intrinsic drug sensitivity, tumor cell stemness, etc.), and/or the prognosis of the tumor (e.g., metastasized, immune-resistant, etc.). Exemplary subtypes of genomics data that may be relevant to tumor development can include, but not limited to genome amplification (as represented genomic copy number aberrations), somatic mutations (e.g., point mutation (e.g., nonsense mutation, missense mutation, etc.), deletion, insertion, etc.), genomic rearrangements (e.g., intrachromosomal rearrangement, extrachromosomal rearrangement, translocation, etc.), appearance and copy numbers of extrachromosomal genomes (e.g., double minute chromosome, etc.). In addition, genomic data may also include tumor mutation burden that is measured by the number of mutations carried by the tumor cells or appeared in the tumor cell in a predetermined period of time or within a relevant time period.

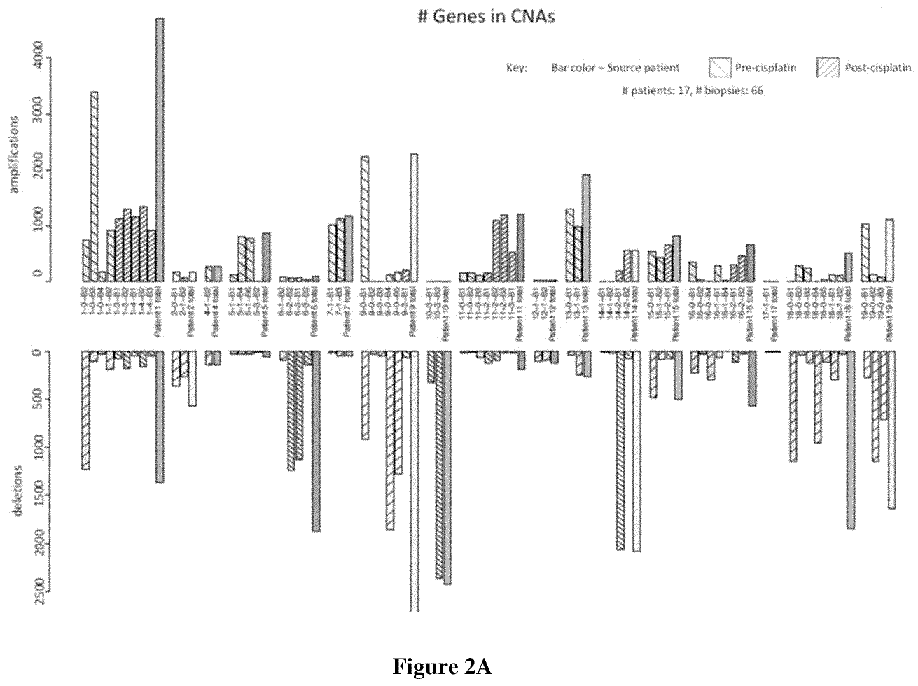

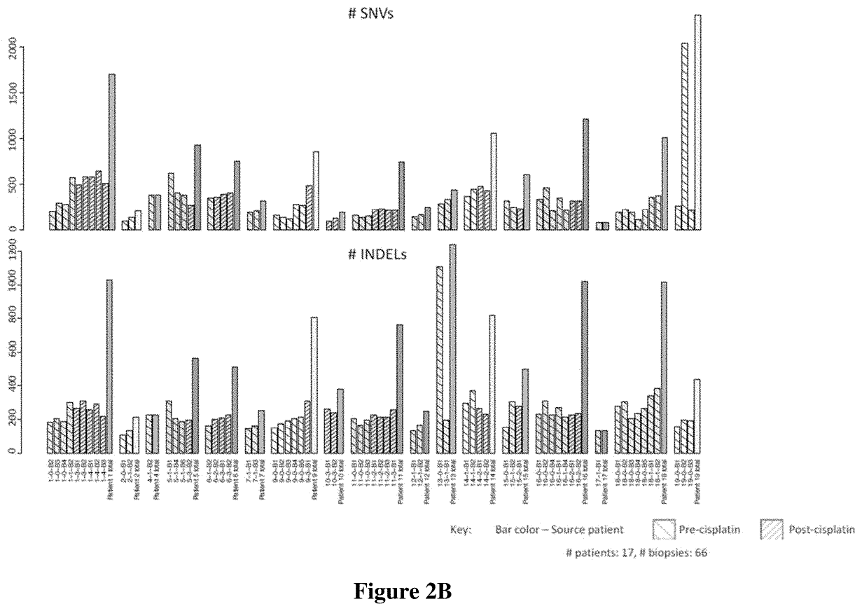

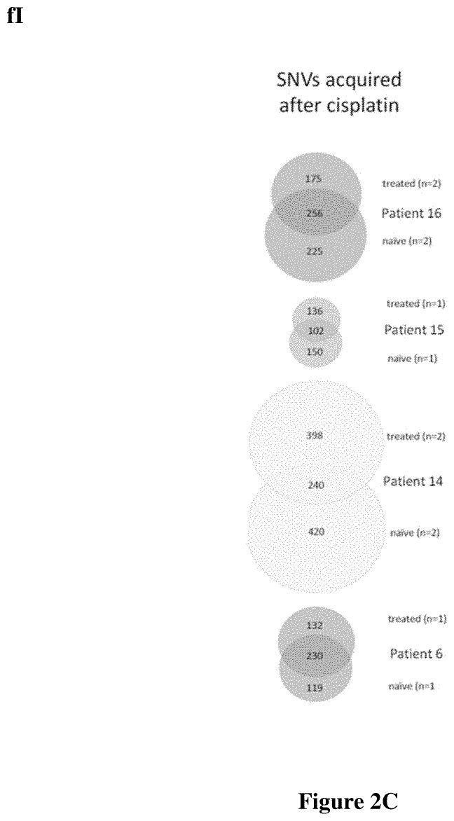

For example, FIGS. 2A-C shows tumor mutation burden identified in 66 biopsies obtained from 17 metastatic triple negative breast cancer patients who were naïve to the platinum-based treatment and received cisplatin during the trial. FIG. 2A illustrates the number of genes overlapping a highly-confident genomic copy-number aberration (CNA) in each individual biopsy, and the total number of genes with a CNA for each patient before and after cisplatin treatment. A genomic amplification is defined as a genomic region >5 kb with a median copy number estimate above 6 copies and a genomic deletion is defined as having a median copy number estimate below 1 copy. Amplification events detected in the genome are plotted in the upper bar graphs, and deletion events detected in the genome are plotted in lower bar graphs. Copy Number estimates were derived from relative coverage as reported by circular binary segmentation (CBS) and tumor purity/ploidy estimates. The inventors observed that some individual metastases undergo relatively numerous CNA events. Lightly hatched (top left to bottom right) is before cisplatin treatment and densely hatched (bottom left to top right) is after cisplatin treatment.

FIG. 2B illustrates the number of confident exomic single nucleotide variants (SNVs) (above) and insertion and deletion of bases (INDELs) (below) seen in each biopsy from 17 metastatic triple negative breast cancer patients, and the total number of SNVs & INDELs per patient. SNVs and INDELs were detected by comparing the exome sequence data obtained from the tumor cells and matched normal tissues as described above. So identified mutations are further processed by applying several filtering steps to generate confident calls for mutation (a genuine somatic mutation present in the genome or exome other than mere sequencing error, germline mutation, or allele variation, etc.). Thus, exemplary filtering may be based on the number of read supports, base quality, single nucleotide polymorphism (SNP) that can be identified from the single nucleotide polymorphism database (dbSNP) and somatic vs. germline variant calling. The inventors found that TNBC patients in this study tend to slowly acquire SNVs and INDELs counts in later biopsies. Yet, initial mutational burden after cisplatin treatment differs significantly (up to 10 folds in mutational burden magnitude) between patients, indicating that the tumor mutational burden can be a patient-specific factor to affect the intrinsic properties of the tumor tissue or tumor cell in response to the cisplatin treatment.

In order to confirm the effect of cisplatin treatment to the tumor mutation burden, biopsied tumor tissues were obtained from four patients immediately prior to cisplatin therapy and after treatment. Whole genome sequencing data was obtained from each of the biopsied tissue and the number of SNVs obtained before and after cisplatin treatment was analyzed. FIG. 2C depicts the counts of existing and acquired SNVs in the pre-cisplatin and post-cisplatin biopsies. The SNVs arising after the cisplatin treatment may indicate tumor evolution in response to cisplatin treatment (e.g., acquired susceptibility or resistance, etc.). Following cisplatin treatment, the expressions of over 300 genes were significantly perturbed, and 270 mutations on average, per patient were introduced.

In addition to the genomics data, one or more subtypes of transcriptomics data can be used to determine the molecular profile or a molecular signature of the tumor tissue. Exemplary transcriptomics data includes, but not limited to, expression levels of a plurality of mRNAs as measured by quantities of the mRNAs, maturation levels of mRNAs (e.g., existence of poly A tail, etc.), and/or splicing variants of the transcripts. The number of genes (at least two, at least five, at least ten, at least fifteen, etc.), types of transcripts or RNAs (mRNA, miRNA, etc.), or the selection of genes to determine the molecular profile or a molecular signature of the tumor tissue may vary based on the type of tumor, based on the desired information (e.g., information on intrinsic drug sensitivity, tumor cell stemness, etc.), and/or the prognosis of the tumor (e.g., metastasized, immune-resistant, etc.). For example, the selection of genes and/or the number of genes to determine molecular signature related to tumor stemness may differ, or minimally overlap with the selection of genes and/or the number of genes to determine molecular signature related to cell sensitivity to a specific chemotherapeutic drug. It is contemplated that the genes to be included in the relevant transcriptomics data set to differentiate the tumor samples (from the matched normal or among the tumor samples having different physiological characteristics) may include any tumor-specific genes, inflammation-related genes, DNA repair-related genes (e.g., Base excision repair, Mismatch repair, Nucleotide excision repair, Homologous recombination, Non-homologous end-joining, etc.), genes associated with sensitivity to DNA damaging agents, DNA replication machinery-related genes. Yet, it is also contemplated that the genes to be included in the relevant transcriptomics data set to differentiate the tumor samples may include genes not associated with a disease (e.g., housekeeping genes), including, but not limited to, those related to transcription factors, RNA splicing, tRNA synthetases, RNA binding protein, ribosomal proteins, or mitochondrial proteins, or noncoding RNA (e.g., microRNA, small interfering RNA, long non-coding RNA (lncRNA), etc.).

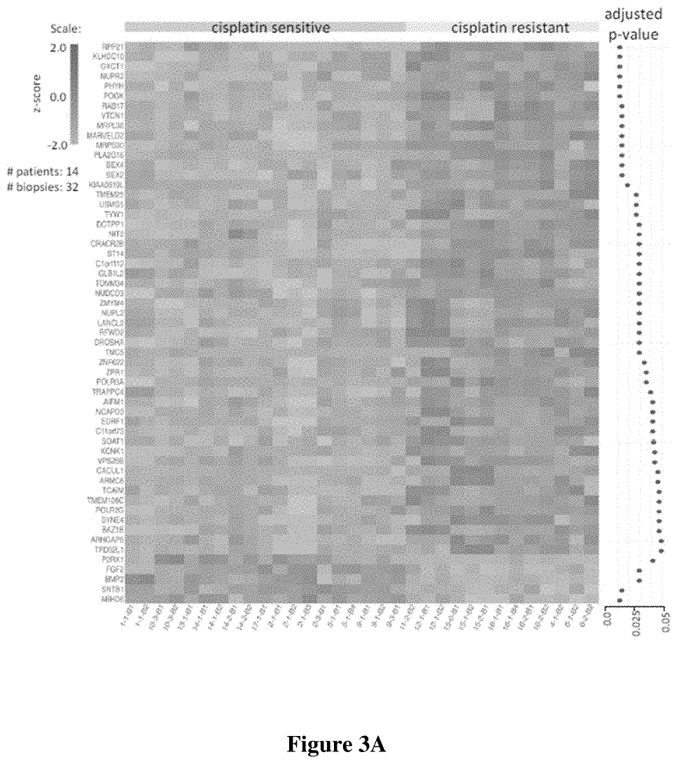

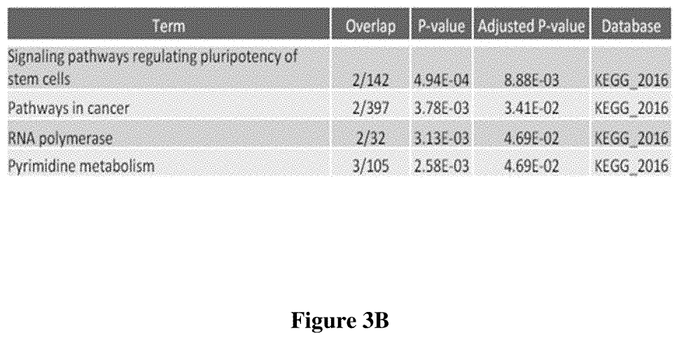

FIG. 3A provides one exemplary RNA expression profiling of 57 genes (shown as a heat map) that differentially express among cisplatin sensitive tumor cells compared to cisplatin resistant tumor cells (q<0.05 using Benjamini-Hochberg adjustment, p<0.05 in adjusted P value). In this example, total 32 biopsied tumor tissues were obtained from 14 patients (8 cisplatin-sensitive patients and 6 cisplatin-resistant patients) and transcriptomics data of those 32 biopsied tumor tissues were compared to identify genes that are differentially expressed in cisplatin sensitive tumor cells and cisplatin resistant tumor cells. The expression levels are quantified and represented as Z-score (z=(expression in tumor sample—mean expression in reference sample)/standard deviation of expression in reference sample). The inventors found that expression level of transcript derived from genes RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, and ABHD6 are significantly different among cisplatin-sensitive and cisplatin-resistant tumor samples. Among the 57 genes, expression level of transcripts derived from RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, and TPD52L1 are higher among cisplatin-sensitive tumor samples, and expression level of transcripts derived from P2RX1, FGF2, BMP2, SNTB1, and ABHD6 are lower among cisplatin-sensitive tumor samples compared to cisplatin-resistant tumor samples. Thus, the inventors contemplate that RNA expression profiles to determine the molecular profiles or signatures of the metastatic TNBC cells in relation to cisplatin sensitivity or resistance can include at least 5 genes, at least 10 genes, at least 15 genes, at least 20 genes, or at least 25 genes among 57 genes identified above.

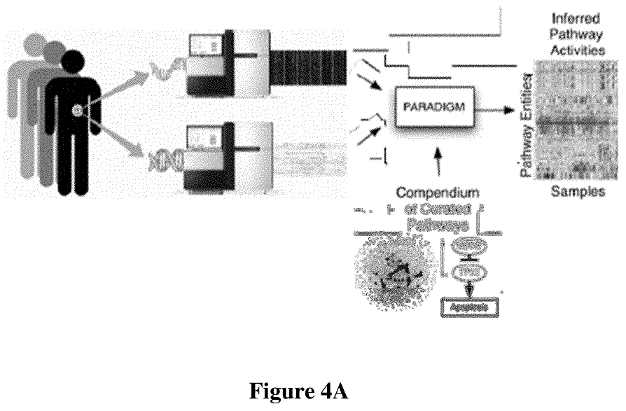

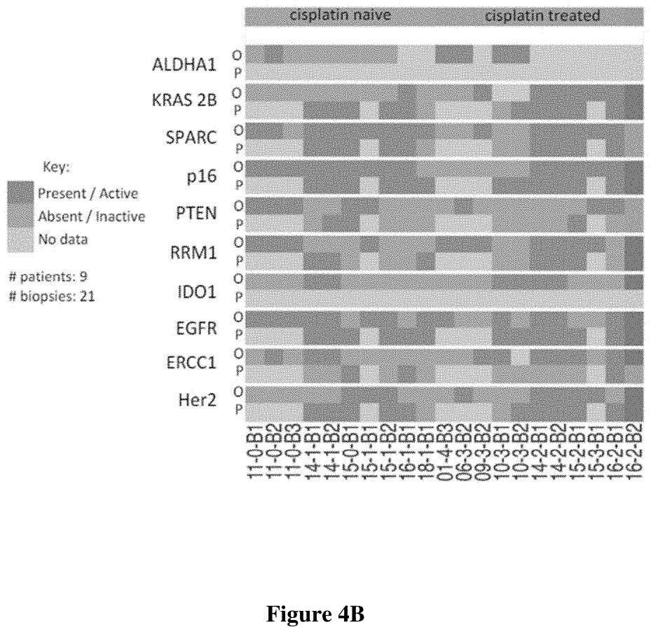

Optionally, one or more subtypes of proteomics data can be used to determine the molecular profile or a molecular signature of the tumor tissue. Exemplary proteomics data includes, but not limited to, quantities of one or more proteins or peptides, post-translational modification of one or proteins or peptides (e.g., phosphorylation, glycosylation, forming a dimer, ubiquitination, etc.), and/or subcellular localization of the proteins or peptides. For example, FIG. 4B shows one exemplary proteomics profiling of 10 proteins (ALDHA1, KRAS2B, SPARC, p16, PTEN, RRM1, IDO1, EGFR, ERCC1, Her2), whose expression levels significantly discriminate cisplatin-native tumor sample from cisplatin treated sample (by Fisher's exact test). Thus, the inventors contemplate that the proteomics profiling to determine the molecular profiles or signatures of the metastatic TNBC cells in relation to cisplatin sensitivity or resistance can include at least one, at least two, at least five, or at least seven proteins listed above. Alternatively and more preferably, the inventors contemplate that the proteomics profiling of the tumor cells can be used as a corroborative data to refine pathway analysis described below. For example, an inferred pathway activity that is not matched from the measured proteomics data (e.g., inferred pathway activity indicates active or increased quantity after cisplatin treatment while measured proteomics data shows repressed or decreased quantity after cisplatin treatment, etc.) can be filtered out or removed from the data set.

### Pathway Analysis

Without wishing to be bound by any specific theory, the inventors contemplate that the mutational profiles and/or the RNA expression profiles of the tumor tissue, either independently or collectively, affect the intracellular signaling networks, which consequently may change the intrinsic properties of the tumor tissues or cells. Thus, in one preferred aspect of the inventive subject matter, so determined mutational profiles and/or the RNA expression profiles of the tumor tissue can be integrated into a pathway model to generate a modified pathway or the tumor-specific pathway. Most typically, the pathway model comprises a plurality of pathway elements (e.g., proteins) that are connected by one or more regulatory nodes. For example, a pathway model [A] is a factor-graph-based pathway model (e.g., PARADIGM pathway model) that comprises pathway elements A, B, and C connected by a regulatory node I between the elements A and B, and another regulatory node II between the element B and C (A-I-B-II-C). The regulatory node I and II represent any factors other than A or B that may affect the activity of B and C. Thus, the pathway model [A] may be coupled to another pathway model [B] via one of the regulatory nodes I and II. Thus, in some embodiments, the pathway model may include a single pathway (e.g., PKA mediated apoptosis pathway, etc.). Consequently, in some embodiments, the pathway model may be a single degree model that includes one or more signaling pathways that are parallel or substantially independent from each other. In other embodiments, the pathway model may be a multi-degree model that may include a plurality of signaling pathways that are coupled via one or more regulatory nodes (e.g., two degree model having pathways [A] and [B] where pathways [A] and [B] are coupled in a regulatory node of the pathway [A], three degree model having pathways [A], [B], and [C] where the pathways [A] and [B] are coupled in a regulatory node of the pathway [A] and pathways [B] and [C] are coupled in a regulatory node of the pathway [B].

The pathway element activity of each pathway element can be inferred or calculated using the omics data as inputs in the central dogma module (DNA-RNA-protein-protein activity) as described in WO 2014/193982, which is incorporated by reference herein. For example, where the gene encoding protein A carries multiple genomic mutations in the exome, and RNA expression level of the gene increase upon a drug treatment, it can be inferred from such genomics and transcriptomics profile, the quantity of the protein may be increased while the activity of such protein may provide a dominant negative effect in the signaling pathway (where protein A is an element of the signaling pathway) due to missense mutations in the critical post-translational modification residues. Based on such inferred individual pathway element activity, the activity of downstream signaling pathway element can be inferred in the same signaling pathway or another signaling pathway that is connected by a regulatory node.

Consequently, diverse types of omics data can be integrated into a single pathway model to so allow on the basis of measured attributes (e.g., DNA copy number and/or mutations, RNA transcription level, protein quantities and/or activities) calculation of inferred attributes (e.g., DNA copy number and/or mutations, RNA transcription level, protein quantities and/or activities for which no data were obtained from the sample) and also calculation of inferred pathway activities. Advantageously, such calculations can employ the entirety of available omics data, or only use omics data that have significant deviations from corresponding normal values (e.g., due to copy number changes, over- or under-expression, loss of protein activity, etc.). Using such system, it should be appreciated that instead of analyzing only single or multiple markers, cell signaling activities and changes in such signaling pathways can be detected that would otherwise be unnoticed when considering only single or multiple markers in disregard of their function.

Preferably, the pathway models can be pre-trained via a machine learning algorithms (e.g., Linear kernel SVM, First order polynomial kernel SVM, Second order polynomial kernel SVM, Ridge regression, Lasso, Elastic net, Sequential minimal optimization, Random forest, J48 trees, Naive bayes, JRip rules, HyperPipes, and NMFpredictor) with omics data from the healthy individuals as inputs and corroborative data. In such embodiment, through the machine learning algorithms, each pathway element and the factor to the regulatory node will be provided with weights and directions to determine the activity of the downstream pathway elements. For example, where the pathway elements A and B are connected to regulatory node I, each, or at least one of quantity (e.g., copy number, expression level of RNA) and/or status (e.g., types and locations of mutations, number of phosphorylation for phosphorylated protein, etc.) of pathway element A and/or any factors of regulatory node I (e.g., activity of an enzyme affecting the activity of pathway element A, etc.) are integrated or calculated to infer the activity of pathway element B (e.g., quantity, status of protein B).

Consequently, such trained pathway model can be used as a template to predict how the pathway or pathway elements would be changed in the tumor tissue. As depicted in FIG. 4A, omics data obtained from the patient (and preferably compared with the matched normal tissue or healthy tissue from healthy individuals) can be integrated into a factor-graph-based model using PARADIGM to infer or predict which and how pathway elements would be changed due to the tumor-specific omics data changes compared to the compared with the matched normal tissue or healthy tissue from healthy individuals. While FIG. 4A depicts PARADIGM as an exemplary pathway analysis method and/or tool using omics data, it should be noted that any suitable pathway models that can be machine-trained and produce reliable output data as such are deemed appropriate. Thus, suitable pathway models include Gene Set Enrichment Analysis (GSEA, Broad Institute) based models, Signaling Pathway Impact Analysis (SPIA, Bioconductor) based models, and PathOlogist pathway models (NCBI) as well as factor-graph based models, and especially PARADIGM as described in WO2011/139345A2, WO2013/062505A1, and WO2014/059036, all incorporated by reference herein.

The numbers and types of signaling pathways and/or the scope of cellular networks that may be relevant or closely related to the prognosis of tumor may vary depending on the type of tumor, stages of the tumor, and/or analysis interest (e.g., drug sensitivity, tumor cell stemness, immune resistance, etc.). In other words, numbers and types of signaling pathways to be analyzed using the omics data may be limited to those that were predetermined to be relevant or to those identified to be most relevant or most affected by changes in omics data. Thus, in one embodiment, the signaling pathways may be selected based on the number of signaling pathway elements and/or degree of impact on the signaling pathway elements in the signaling pathways represented by differential expression of genes in the tumor samples. For example, the inventors found that, as shown in FIG. 3B, many genes showing differential expression levels among cisplatin sensitive and resistant tumors are pathway elements of several signaling pathways (e.g., signaling pathways regulating pluripotency of stem cells, cancer pathways, RNA polymerase regulation pathway, pyrimidine metabolism pathway), indicating that those signaling pathways may be modified upon changes in mutational and/or expression profiles of the genes that are elements of the signaling pathways.

Some signaling pathways may be substantially affected by the change of a signaling pathway element even if the signaling pathway element is not present in those signaling pathways. Thus, alternatively and/or additionally, numbers and types of signaling pathways may be selected based on the overall impact on the pathways due to the changes in the omics data. In such embodiments, a plurality of signaling pathways that may present in the tumor cell can be examined using machine learning algorithms and one or more variable factors (e.g., differentially expressed genes as shown in FIG. 3A), and the most highly impacted signaling pathway by the changes in the omics data can be selected.

An exemplary prediction of pathway element activity in the central dogma module (DNA-RNA-protein-protein activity) in TNBC patient's tumors are shown in FIG. 4B that depicts measured protein amounts (0) from the tumor sample using quantitative mass-spectroscopy and inferred protein activity from PARADIGM (P) of top 10 proteins (ALDHA1, KRAS2B, SPARC, p16, PTEN, RRM1, IDO1, EGFR, ERCC1, Her2) that distinguish cisplatin treated tumor samples from cisplatin naïve tumor samples (by Fisher's exact test). “Absence/Inactive” activity of protein is defined as a protein quantification in the bottom 10% of all observed values from >3700 clinical samples. Most of the proteins that are observed via quantitative mass-spectroscopy as having distinguishing expression level between the cisplatin-naïve and cisplatin-treated samples also presented similar distinguishing feature in the inferred protein activity via PARADIGM analysis. Thus, the accuracy of pathway analysis predicting the protein activity could be cross checked using the measured protein activity from the same tumor sample.

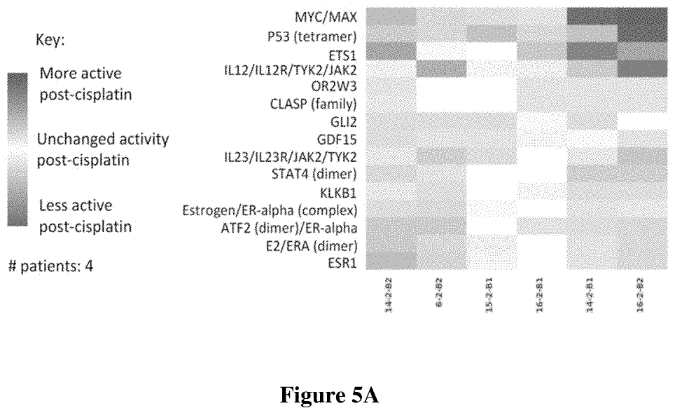

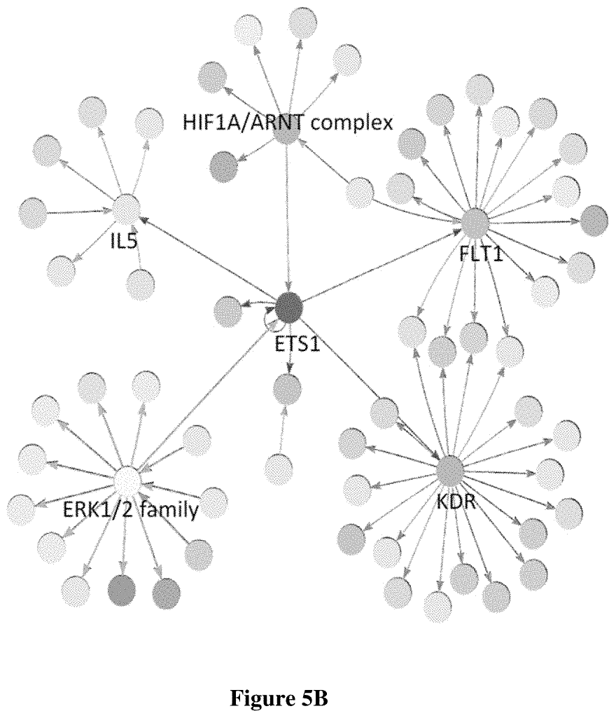

Such obtained genomic mutation profile, RNA expression profile, and optionally proteomic profiling (either measured from the sample or inferred by pathway analysis) can be further used collectively to identify or predict signaling pathway elements in the relevant signaling pathway that are most significantly changed in the tumor tissue. For example, FIG. 5A shows a heat map of 15 proteins or protein complexes that shows most changes upon cisplatin treatment. As shown MYC/MAX and p53 activities were predicted to be more active after cisplatin treatment, and ETS1 activity was predicted to be most repressed after cisplatin treatment.

So predicted or inferred protein activity can be further used to infer how the activity of the signaling pathway, overall, or even the signaling networks comprising a plurality of signaling pathways is changed or modified in response to an event (e.g., drug treatment, etc.). In other words, where the pathway model includes a single signaling pathway and the inferred protein activity of a sole repressor of the signaling pathway is enhanced, the signaling pathway activity, overall, can be predicted to be downregulated or repressed. Also, where two or more signaling pathways, each of which includes a plurality of signaling pathway elements, the individual signaling pathway element's activity can be used to predict other pathway elements in the same pathway or another pathways that are affected by the individual pathway element. For example, FIG. 5B shows a multi-degree signaling network model that two or more signaling pathways are connected directly or indirectly (e.g., ETS1 pathway and FLT1 pathway are connected directly via ETS1 and FLT1, and at the same time, ETS1 pathway and FLT1 pathway are indirectly connected via HIF1A/ARNT complex or KDR pathway) suppressed activity of ETS1 is predicted to suppress other signaling pathways or pathway elements, including, IL-5, FLT1, ERK1/2, KDR, and HIF1A/ARNT complex, which are further predicted to suppress or de-suppress the downstream signaling pathway elements in their signaling pathways.

The inventors further contemplate that modified pathway activity in the tumor cell occurred upon an event can be an indicative of a response to the event. For example, where the event is an anti-tumor treatment to the tumor cell, the response to the event may include increasing sensitivity or susceptibility to the anti-tumor treatment, developing or acquiring resistance to the anti-tumor treatment, or unresponsiveness to the anti-tumor treatment. In some embodiments, the modified pathway activities in the tumor cell can be scored to determine the likelihood of the response to the event. In such embodiments, each modified pathway activity (e.g., each modified protein expression level or post-translational modification of the pathway element, etc.) can be scored based on the degree of changes (e.g., significantly increased or decreased, moderately increased or decreased, etc.), and/or the number of downstream elements or other signaling pathways that are affected by the modified pathway activity, and/or degree of signal amplification (e.g., the number of downstream signal pathway elements sending feedback signal to the pathway element of the modified pathway activity, etc.), and/or degree of overall activity change, as a whole, due to the modified pathway activity. Such scored modified pathway activities can be associated with the response when the score is above or below predetermined threshold (e.g., likely acquire resistance to the anti-tumor treatment when the score is above the threshold, and likely remain susceptible or sensitive to the anti-tumor treatment when the score is below the threshold, etc.).

It is further contemplated that the inference of modified pathway activities in the tumor can be used to predicting treatment efficacy of an anti-tumor treatment on a metastatic tumor in a patient. Most typically, a plurality of tumor sample from at least two different anatomical locations can be obtained from the patient and omics data sets (e.g., genomics data, transcriptomics data, proteomics data) can be derived from each of the plurality of tumor samples as described above. For each tumor sample, genomic mutation profile (e.g., a mutation burden of a plurality of somatic mutations) and RNA expression profiles are determined and such determined profiles are integrated into a pathway model to infer modified pathway activities in each tumor sample. It is contemplated that different modified pathway activities calculated from two tumor samples from two different anatomical locations may indicate modified intrinsic properties of the tumors, likely upon metastasis, which further may lead to different sensitivity and/or resistance to the anti-tumor treatment.

Thus, the inventors further contemplate that, based on the modified pathway activities, and preferably based on the score derived from the modified pathway activities, a patient's record can be generated or updated, a new treatment plan can be recommended, or a previously used treatment plan can be updated. For example, where the metastasized tissue shows modified pathway activities that indicates acquired resistance to one type of anti-tumor treatment, the patient's record may include information and/or recommendation to administer other type of anti-tumor treatment (e.g., immune therapy, QUILT based combinatorial therapy, etc.).

It should be appreciated that the inventive subject matter uses comprehensive molecular profiles obtained from multiple aspects of the omics data set to increase the confidence in associating the molecular profile to the physiological properties of the tumor tissue. Further, such multiple aspects of the omics data sets are integrated to determine modified signaling pathway elements, modified signaling pathway activities, and further modified signaling pathway networks that may affect the cell's intrinsic properties. This approach allows more accurate prediction of cell's intrinsic properties as individual variances in multiple aspects of omics data can be collectively considered. Further, it should be also noted that the inventive subject matter uses only relevant signaling pathway models to be modified with omics data depending on the type of tumor and type of information desired to obtain. Such approach removes further filtering step of information obtained from gigantic amount of pathway analysis with omics data to refine to relevant data such that the overall data processing of pathway analysis becomes quick and efficient.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of predicting prognosis of a tumor in a patient upon an anti-tumor treatment, comprising:
obtaining a tumor sample from the patient and an omics data set from the tumor sample;
determining a genomic mutation profile from the omics data set, wherein the genomic mutation profile comprises a mutation burden of a plurality of somatic mutations;
determining an RNA expression profile of a plurality of genes;
obtaining a pathway model comprising a plurality of pathway elements;
inferring a modified pathway activity by integrating the genomic mutation profile and the RNA expression profile into the pathway model;
wherein at least one of the genomic mutation profile and the RNA expression profile is related to at least one of the pathway elements; and
wherein the modified pathway activity is an indicative of a response to the anti-tumor treatment.

2. The method of claim 1, wherein the tumor is a triple-negative breast cancer, and the anti-tumor treatment is cisplatin.

3. The method of any of preceding claims, wherein the plurality of somatic mutations comprises a point mutation, an amplification, a deletion, and an insertion.

4. The method of any of preceding claims, wherein the mutational burden is determined by number of the plurality of somatic mutations occurring in a predetermined time period.

5. The method of any of preceding claims, wherein at least two of the plurality of genes are selected from a group consisting of RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6.

6. The method of any of preceding claims, wherein at least two of the plurality of genes are involved in at least one of a signaling pathway regulating pluripotency of stem cells, a cancer signaling pathway, and a pyrimidine metabolism.

7. The method of any of preceding claims, wherein the genomic mutation profile is obtained from a whole genome sequencing, an exome sequencing, or RNAseq.

8. The method of any of preceding claims, wherein the genomic mutation profile further comprises a copy number of a genome segment having the somatic mutation.

9. The method of any of preceding claims, wherein the modified pathway activity is at least one of modified protein expression level and modified post-translational modification of at least one pathway element.

11. The method of any of preceding claims, wherein the modified pathway activity comprises a pathway element in MYC/MAX pathway, p53 pathway or ETS1 pathway.

12. The method of any of preceding claims, wherein the modified pathway activity is inferred using PARADIGM pathway analysis.

13. The method of any of preceding claims, wherein the response to the treatment comprises sensitivity, unresponsiveness, and acquired resistance to the treatment.

14. The method of any of preceding claims, further comprising:
measuring a protein activity from the tumor sample, wherein the protein activity comprises at least one of quantity of the protein and a post-translational modification of the protein; and
comparing the protein activity with the modified pathway activity.

15. The method of claim 14, further comprising integrating the protein activity to refine the modified pathway activity.

16. The method of any of preceding claims, further comprising generating or updating the patient's record using the modified pathway activity.

17. The method of claim 1, wherein the plurality of somatic mutations comprises a point mutation, an amplification, a deletion, and an insertion.

18. The method of claim 1, wherein the mutational burden is determined by number of the plurality of somatic mutations occurring in a predetermined time period.

19. The method of claim 1, wherein at least two of the plurality of genes are selected from a group consisting of RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6.

20. The method of claim 1, wherein at least two of the plurality of genes are involved in at least one of a signaling pathway regulating pluripotency of stem cells, a cancer signaling pathway, and a pyrimidine metabolism.

21. The method of claim 1, wherein the genomic mutation profile is obtained from a whole genome sequencing, an exome sequencing, or RNAseq.

22. The method of claim 1, wherein the genomic mutation profile further comprises a copy number of a genome segment having the somatic mutation.

23. The method of claim 1, wherein the modified pathway activity is at least one of modified protein expression level and modified post-translational modification of at least one pathway element.

24. The method of claim 1, wherein the modified pathway activity comprises a pathway element in MYC/MAX pathway, p53 pathway or ETS1 pathway.

25. The method of claim 1, wherein the modified pathway activity is inferred using PARADIGM pathway analysis.

26. The method of claim 1, wherein the response to the treatment comprises sensitivity, unresponsiveness, and acquired resistance to the treatment.

27. The method of claim 1, further comprising:
measuring a protein activity from the tumor sample, wherein the protein activity comprises at least one of quantity of the protein and a post-translational modification of the protein; and
comparing the protein activity with the modified pathway activity.

28. The method of claim 27, further comprising integrating the protein activity to refine the modified pathway activity.

29. The method of claim 1, further comprising generating or updating the patient's record using the modified pathway activity.

30. A method of predicting treatment efficacy of an anti-tumor treatment on a metastatic tumor in a patient, comprising:
obtaining a plurality of tumor sample from at least two different anatomical locations in the patient and respective omics data sets from each of the tumor sample;
determining respective genomic mutation profiles from the omics data sets, wherein the genomic mutation profile comprises a mutation burden of a plurality of somatic mutations;
determining respective RNA expression profiles of a plurality of genes from each of the tumor sample;
obtaining a pathway model comprising a plurality of pathway elements;
inferring respective modified pathway activities of the tumor samples by integrating the genomic mutation profile and the RNA expression profile into the pathway model;
wherein at least one of the genomic mutation profile and the RNA expression profile is related to at least one of the pathway elements;
wherein the modified pathway activity is an indicative of a response to the anti-tumor treatment; and
generating or updating the patient's record using the modified pathway activities.

31. The method of claim 30, wherein the tumor is a metastatic triple-negative breast cancer, and the anti-tumor treatment is cisplatin.

32. The method of any of claims 30-31, wherein the plurality of somatic mutations comprises a point mutation, an amplification, a deletion, and an insertion.

33. The method of any of claims 30-32, wherein the mutational burden is determined by number of the plurality of somatic mutations occurring in a predetermined time period.

34. The method of any of claims 30-33, wherein at least two of the plurality of genes are selected from a group consisting of RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6.

35. The method of any of claims 30-34, wherein at least two of the plurality of genes are involved in at least one of a signaling pathway regulating pluripotency of stem cells, a cancer signaling pathway, and a pyrimidine metabolism.

36. The method of any of claims 30-35, wherein the genomic mutation profile is obtained from a whole genome sequencing, an exome sequencing, or RNAseq.

37. The method of any of claims 30-36, wherein the genomic mutation profile further comprises a copy number of a genome segment having the somatic mutation.

38. The method of any of any of claims 30-37, wherein the modified pathway activity is at least one of modified protein expression level and modified post-translational modification of at least one pathway element.

39. The method of any of claims 30-38, wherein the modified pathway activity comprises a pathway element in MYC/MAX pathway, p53 pathway or ETS1 pathway.

40. The method of any of claims 30-39, wherein the modified pathway activity is inferred using PARADIGM pathway analysis.

41. The method of any of claims 30-40, further comprising:
measuring a protein activity from the tumor sample, wherein the protein activity comprises at least one of quantity of the protein and a post-translational modification of the protein; and
comparing the protein activity with the modified pathway activity.

42. The method of claim 41, further comprising integrating the protein activity to refine the modified pathway activity.

43. The method of any of claims 30-42, wherein the response to the treatment comprises sensitivity, unresponsiveness, and acquired resistance to the treatment.

44. The method of any of claims 30-43, wherein the updated patient's record comprises at least one of a likelihood of success of the anti-tumor treatment to treat the metastatic tumor and an updated treatment recommendation to the patient.

46. The method of claim 30, wherein the plurality of somatic mutations comprises a point mutation, an amplification, a deletion, and an insertion.

47. The method of claim 30, wherein the mutational burden is determined by number of the plurality of somatic mutations occurring in a predetermined time period.

48. The method of claim 30, wherein at least two of the plurality of genes are selected from a group consisting of RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6.

49. The method of claim 30, wherein at least two of the plurality of genes are involved in at least one of a signaling pathway regulating pluripotency of stem cells, a cancer signaling pathway, and a pyrimidine metabolism.

50. The method of claim 30, wherein the genomic mutation profile is obtained from a whole genome sequencing, an exome sequencing, or RNAseq.

51. The method of claim 30, wherein the genomic mutation profile further comprises a copy number of a genome segment having the somatic mutation.

52. The method of claim 30, wherein the modified pathway activity is at least one of modified protein expression level and modified post-translational modification of at least one pathway element.

53. The method of claim 30, wherein the modified pathway activity comprises a pathway element in MYC/MAX pathway, p53 pathway or ETS1 pathway.

54. The method of claim 30, wherein the modified pathway activity is inferred using PARADIGM pathway analysis.

55. The method of claim 30, further comprising:
measuring a protein activity from the tumor sample, wherein the protein activity comprises at least one of quantity of the protein and a post-translational modification of the protein; and
comparing the protein activity with the modified pathway activity.

56. The method of claim 30, further comprising integrating the protein activity to refine the modified pathway activity.

57. The method of claim 30, wherein the response to the treatment comprises sensitivity, unresponsiveness, and acquired resistance to the treatment.

58. The method of claim 30, wherein the updated patient's record comprises at least one of a likelihood of success of the anti-tumor treatment to treat the metastatic tumor and an updated treatment recommendation to the patient.

59. A method of predicting a response to cisplatin treatment in a patient having a triple negative breast cancer, comprising:
obtaining a tumor sample from the patient and an omics data set from the tumor sample;
determining an RNA expression profile of a plurality of genes, wherein at least one of the plurality of genes are related to at least one of a signaling pathway regulating pluripotency of stem cells, a cancer signaling pathway, and a pyrimidine metabolism;
inferring a modified pathway activity by integrating the RNA expression profile, wherein the RNA expression profile is related to at least one of the pathway elements; and
wherein the modified pathway activity is an indicative of a response to the cisplatin treatment.

60. The method of claim 59, wherein at least two of the plurality of genes are selected from a group consisting of RPP21, KLHDC10, OXCT1, NUPR2, PHYH, POGK, RAB17, BTCN1, MRPL36, MARBELD2, MRPS30, PLA2G16, BEX4, BEX2, KIAA0319L, TMEM25, USMG5, TYW1, DCTPP1, NIT2, CRACR2B, ST14, C1orf112, GLB1L2, TOMM34, NUDCD3, ZMYM4, NUPL2, LANCL2, RFWD2, DROSHA, TMC5, ZNF622, ZPR1, POLR3A, TRAPPC4, AIFM1, NCAPD3, EDRF1, C11orf73, SOAT1, KCNK1, VPS26B, CACUL1, ARMC6, TCAIM, TMEM106C, POLR2G, SYNE4, BAZ1B, ARHGAP8, TPD52L1, P2RX1, FGF2, BMP2, SNTB1, ABHD6.

61. The method of any of claims 59-60, wherein the RNA expression profile is determined by comparing the RNA expression level of the plurality of genes in the tumor sample with RNA expression level of the plurality of genes in the matched normal tissue.

62. The method of any of claims 59-61, further comprising:
determining a genomic mutation profile from the omics data set, wherein the genomic mutation profile comprises a mutation burden of a plurality of somatic mutations; and
inferring a modified pathway activity by integrating the genomic mutation profile and the RNA expression profile into the pathway model.

63. The method of claim 62, wherein the plurality of somatic mutations comprises a point mutation, an amplification, a deletion, and an insertion.

64. The method of claim 62, wherein the mutational burden is determined by number of the plurality of somatic mutations occurring in a predetermined time period.

65. The method of claim 62, wherein the genomic mutation profile is obtained from a whole genome sequencing, an exome sequencing, or RNAseq.

66. The method of claim 62, wherein the genomic mutation profile further comprises a copy number of a genome segment having the somatic mutation.

67. The method of any of claims 59-66, wherein the modified pathway activity is at least one of modified protein expression level and modified post-translational modification of at least one pathway element.

68. The method of any of claims 59-67, wherein the modified pathway activity comprises a pathway element in MYC/MAX pathway, p53 pathway or ETS1 pathway.

69. The method of any of claims 59-68, wherein the modified pathway activity is inferred using PARADIGM pathway analysis.

70. The method of any of claims 59-69, wherein the response to the cisplatin treatment comprises sensitivity, unresponsiveness, and acquired resistance to the cisplatin treatment.

71. The method of any of claims 59-70, further comprising:
measuring a protein activity from the tumor sample, wherein the protein activity comprises at least one of quantity of the protein and a post-translational modification of the protein; and
comparing the protein activity with the modified pathway activity.

72. The method of claim 71, further comprising integrating the protein activity to refine the modified pathway activity.

73. The method of any of claims 59-72, further comprising generating or updating the patient's record using the modified pathway activity.

74. The method of claim 59, wherein the RNA expression profile is determined by comparing the RNA expression level of the plurality of genes in the tumor sample with RNA expression level of the plurality of genes in the matched normal tissue.

75. The method of claim 59, further comprising:
determining a genomic mutation profile from the omics data set, wherein the genomic mutation profile comprises a mutation burden of a plurality of somatic mutations; and
inferring a modified pathway activity by integrating the genomic mutation profile and the RNA expression profile into the pathway model.

76. The method of claim 75, wherein the plurality of somatic mutations comprises a point mutation, an amplification, a deletion, and an insertion.

77. The method of claim 75, wherein the mutational burden is determined by number of the plurality of somatic mutations occurring in a predetermined time period.

78. The method of claim 75, wherein the genomic mutation profile is obtained from a whole genome sequencing, an exome sequencing, or RNAseq.

79. The method of claim 75, wherein the genomic mutation profile further comprises a copy number of a genome segment having the somatic mutation.

80. The method of claim 59, wherein the modified pathway activity is at least one of modified protein expression level and modified post-translational modification of at least one pathway element.

81. The method of claim 59, wherein the modified pathway activity comprises a pathway element in MYC/MAX pathway, p53 pathway or ETS1 pathway.

82. The method of claim 59, wherein the modified pathway activity is inferred using PARADIGM pathway analysis.

83. The method of claim 59, wherein the response to the cisplatin treatment comprises sensitivity, unresponsiveness, and acquired resistance to the cisplatin treatment.

84. The method of claim 59, further comprising:
measuring a protein activity from the tumor sample, wherein the protein activity comprises at least one of quantity of the protein and a post-translational modification of the protein; and
comparing the protein activity with the modified pathway activity.

85. The method of claim 74, further comprising integrating the protein activity to refine the modified pathway activity.

86. The method of claim 59, further comprising generating or updating the patient's record using the modified pathway activity.
